(19) United States
(12) Patent Application Publication
(10) Pub. No.: US 2016/0030703 A1
(43) Pub. Date: Feb. 4, 2016

(54) METHOD AND DEVICE FOR INCREASING HUMAN ABILITY FOR IDEA GENERATION AND INSIGHT RELATED TASKS USING DC STIMULATION

(71) Applicant: Richard Chi, Sydney (AU)

(72) Inventor: Richard Chi, Sydney (AU)

(21) Appl. No.: 14/447,616

(22) Filed: Jul. 31, 2014

(51) Int. Cl. A61M 21/02; A61N 1/24; A61N 1/04

(52) U.S. Cl. CPC A61M 21/02; A61N 1/0456; A61N 1/0476; A61N 1/24; A61M 2021/0055

(57) ABSTRACT

This invention described a method of, and device for increasing human ability for idea generation and insight related tasks. “Thinking outside the box” is difficult and often requires a qualitatively different perception of the task at hand. One aspect of the invention is a method for facilitating such pursuits by applying electrical stimulation at selected brain areas before or during an idea generation or insight related task. One aspect of our method is to temporarily enable a person to perceive insight related tasks in a way that is less influenced by past expectations and more open to novel representations. In one embodiment, this is achieved by suppressing the left anterior temporal lobe and/or facilitating the right anterior temporal lobe with non-invasive electrical stimulation.

This application is a continuation of International Application No. PCT/AU2013/000074, filed Jan. 31 2013, which claims the benefit of Provisional Application No. 61/593,189, filed 31 Jan. 2012.

### FIELD OF INVENTION

This invention relates to enhancing human ability by applying electrical stimulation such as DC stimulation.

### COPYRIGHT & TRADEMARK NOTICES

A portion of the disclosure of this patent document contains material that is subject to copyright protection. The copyright owner has no objection to the facsimile reproduction by any one of the patent document or the patent disclosure, as it appears in the files or records of any patent office in which the disclosure is filed, e.g., the U.S. Patent and Trademark Office, but otherwise reserves all copyrights whatsoever.

Certain marks referenced herein may be trademarks or registered trademarks of third parties. Use of these marks is solely for providing an enabling disclosure by way of example and is not to be construed as limiting the scope of this invention to material associated with such trademarks.

### RELATED DOCUMENTS

Chi R P, Snyder A W (2011) Facilitate Insight by Non-Invasive Brain Stimulation. PLoS ONE 6(2): e16655. doi:10.1371/journal.pone.0016655 Editor: Dorothy Bishop, University of Oxford, United Kingdom, published 2 Feb. 2011, incorporated herein in the form as the Appendix, and forming part of the description of the present invention. This document is referred to herein as interchangeably as Chi and Snyder (2011) and “Chi and Snyder 2011”. The document is also available online at http://www˜dot˜plosone˜dot˜org/article/info%3Adoi%2F10˜dot˜1371% 2Fjournal˜dot˜pone˜dot-0016655, (retrieved 30 Jan. 2012), wherein ˜dot˜ represents the period (“.”) character in the actual URL/Web address.

### REFERENCES

The following references are cited herein:

Baron S G, Osherson D (2011) Evidence for conceptual combination in the left anterior temporal lobe. Neuroimage 55:1847-1852.

Bell V, Reddy V, Halligan P, Kirov G, Ellis H (2007) Relative suppression of magical thinking: a transcranial magnetic stimulation study. Cortex 43:551-557.

Boggio P S, Castro L O, Savagim E A, Braite R, Cruz V C, Rocha R R, Rigonatti S P, Silva M T A, Fregni F (2006) Enhancement of non-dominant hand motor function by anodal transcranial direct current stimulation. Neurosci Lett 404:232-236.

Chi R P, Snyder A W (2011) Facilitate Insight by Non-Invasive Brain Stimulation. PLoS ONE 6:181-197.

Chi R P, Fregni F, Snyder A W (2010) Visual memory improved by non-invasive brain stimulation. Brain Res 1353:168-75.

Chronicle E P, Ormerod T C, MacGregor J N (2001) When insight just won't come: The failure of visual cues in the nine-dot problem. Q J Exp Psychol 54:903-919.

Clark V P, Coffman B A, Mayer A R, Weisend M P, Lane T D R, Calhoun V D, Raybourn E M, Garcia C M, Wassermann E M (2010) TDCS Guided using fMRI Significantly Accelerates Learning to Identify Concealed Objects. Neuroimage 59:117-128.

Cohen Kadosh R, Soskic S, Iuculano T, Kanai R, Walsh V (2010) Modulating Neuronal Activity Produces Specific and Long-Lasting Changes in Numerical Competence. Curr Biol 20:2016-2020.

Datta A, Bansal V, Diaz J, Patel J, Reato D, Bikson M (2009) Gyri-precise head model of transcranial direct current stimulation: improved spatial focality using a ring electrode versus conventional rectangular pad. Brain Stimul 2:201-207.

Gandiga P, Hummel F, Cohen L (2006) Transcranial DC stimulation (tDCS): a tool for double-blind sham-controlled clinical studies in brain stimulation. Clin Neurophysiol 117:845-850.

Gazzaniga M S (2002) The split brain revisited. Sci Amer 12:27-31.

Goldberg E (2009) The new executive brain: frontal lobes in a complex world: Oxford University Press.

Hamilton R, Messing S, Chatterjee A (2011) Rethinking the thinking cap. Neurology 76:1726-1734.

Kahneman D, Slovic P, Tversky A (1982) Judgment under uncertainty: Heuristics and biases: Cambridge Univ Press.

Kershaw T C, Ohlsson S (2004) Multiple Causes of Difficulty in Insight: The Case of the Nine-Dot Problem. J Exp Psychol Learn 30:3-13.

Miller B L, Cummings J, Mishkin F, Boone K, Prince F, Ponton M, Cotman C (1998) Emergence of artistic talent in frontotemporal dementia. Neurology 51:978-982.

Nitsche M, Cohen L, Wassermann E, Priori A, Lang N, Antal A, Paulus W, Hummel F, Boggio P, Fregni F (2008) Transcranial direct current stimulation: State of the art 2008. Brain Stimulation 1:206-223.

Paquette C, Sidel M, Radinska B A, Soucy J P, Thiel A (2011) Bilateral transcranial direct current stimulation modulates activation-induced regional blood flow changes during voluntary movement. J Cereb Blood Flow Metab 31: 2086-2095.

Pascual-Leone A (2010) Get Better at Math by Disrupting Your Brain. Sci Am.

Sack A, Camprodon J, Pascual-Leone A, Goebel R (2005) The dynamics of interhemispheric compensatory processes in mental imagery. Science 308:702-704.

Sadleir R J, Vannorsdall T D, Schretlen D J, Gordon B (2010) Transcranial direct current stimulation (tDCS) in a realistic head model. Neuroimage 51:1310-1318.

Schambra H M, Abe M, Luckenbaugh D A, Reis J, Krakauer J W, Cohen L G (2011) Probing for hemispheric specialization for motor skill learning: a transcranial direct current stimulation study. J Neurophysiol 106:652-661.

Segal, E. (2004). Incubation in insight problem solving. Creativity Research Journal, 16, 141-148.

Snyder A (2009) Explaining and inducing savant skills: privileged access to lower level, less-processed information. Trans R Soc B: Biol Sci 364:1399-1405.

Snyder A, Bahramali H, Hawker T, Mitchell D J (2006) Savant-like numerosity skills revealed in normal people by magnetic pulses. Perception 35:837-845.

Snyder A W, Mitchell D J (1999) Is integer arithmetic fundamental to mental processing?: The mind's secret arithmetic. Proc Roy Soc B: Biol Sci 266:587-592.

Sparing R, Thimm M, Hesse M, Kust J, Karbe H, Fink G (2009) Bidirectional alterations of interhemispheric parietal balance by non-invasive cortical stimulation. Brain 132:3011-3020.

Torrance, E. P. (1979). An instructional model for enhancing incubation, Journal of Creative Behavior, 13, 23-25.

Torrance, E. P., Ball, O. E., and Safter, H. T. (1992). Torrance Tests of Creative Thinking: Streamline scoring guide figural A and B. Scholastic Testing Service, Inc., Bensenville, Ill.

Treffert D (2009) The savant syndrome: an extraordinary condition. A synopsis: past, present, future. Philos Trans R Soc B: Biol Sci 364:1351-1357.

Treffert D A (2010) Islands of genius: the bountiful mind of the autistic, acquired, and sudden savant: Jessica Kingsley Pub.

Turkeltaub P E, Benson J, Hamilton R H, Datta A, Bikson M, Coslett H (in press) Left lateralizing transcranial direct current stimulation improves reading efficiency. Brain Stimulation.

Vines B, Cerruti C, Schlaug G (2008) Dual-hemisphere tDCS facilitates greater improvements for healthy subjects' non-dominant hand compared to uni-hemisphere stimulation. BMC Neurosci 9:103.

Weisberg R W, Alba J W (1981) An examination of the alleged role of “fixation” in the solution of several “insight” problems. J Exp Psychol Gen 110:169-192.

Zheng X, Alsop D C, Schlaug G (2011) Effects of transcranial direct current stimulation (tDCS) on human regional cerebral blood flow. Neuroimage 58:26-33.

### BACKGROUND

Tasks involving what commonly is understood to be “thinking outside the box” are difficult. Once humans have learned to solve problems by one method, humans often have difficulties in generating solutions involving a different kind of insight (see FIG. 4) (Ollinger et al. 2008). One possible reason for this so called “mental set” effect is that our minds are predisposed to observe the world ‘top-down’, through the filters of preconceptions (Gregory, 1980; Snyder, 1999; 2004;). It is theorized that humans do not have a literal view of the world (Bartlett 1932; Snyder 1999). Instead, it is theorized that what humans perceive is automatically shaped by past experiences (Snyder 2004). For example, it is theorized that information consistent with human expectations is often accepted at face value, whereas inconsistent evidence is discounted by the human or hidden from human conscious awareness (Gilovich, 1991). While it is theorized that this “top-down” mechanism helps humans in efficiently dealing with incomplete information in a familiar situation, it is theorized that it can lead to certain cognitive biases and prevent us from reaching better solutions in a different or unfamiliar context (Bilali et al. 2010).

In other words, it is theorized that there is normally a cognitive tradeoff between the necessity of being fast at the familiar on one hand and being receptive to novelty on the other. It would be beneficial if in certain situations humans have an artificial method that can modulate this tradeoff to human advantage. For example, diminishing the so-called ‘top-down’ mechanism so that humans can be less susceptible to the ‘mental set’ effect and more receptive to novel representations during idea generation or an insight related task.

DC stimulation, commonly known as transcranial direct current stimulation (tDCS), is a century old, safe, non-invasive brain stimulation technique that has experienced a recent revival in neuro-rehabilitation and cognitive neuroscience (Nitsche 2008). The technique involves applying a weak direct current (1-2 ma) to the scalp of a person via two saline-soaked sponge electrodes (typically 25-35 cm2), thereby polarizing the underlying brain tissue with electrical fields (Nitsche 2008). It has been shown that such DC stimulation can modulate cortical excitability and spontaneous firing activities in the stimulated region by shifting the resting membrane potential (Nitsche, et al., 2003a). Depending on the polarity of the current flow, cortical excitability can be increased due to sub-threshold membrane depolarization (anodal stimulation) or decreased due to membrane hyperpolarisation (cathodal stimulation) during and beyond the period of stimulation (Nitsche & Paulus, 2000; Nitsche & Paulus, 2001; Nitsche, et al., 2008). A recent study also showed that bilateral DC stimulation at the temporal lobes could modulate hemisphere balance, leading to a greater left lateralization (Turkeltaub et al., 2011).

A clue for achieving this may come from people with brain dysfunctions (Snyder Mitchel 1999; Snyder, 2009; Treffert, 2009) who sometimes paradoxically exhibit islands of genius. For example, Treffert (2009) showed that extraordinary skills in autistic savants are often associated with left hemisphere inhibition together with right hemisphere facilitation. Miller (1998) also found that artistic talent can sometimes emerge spontaneously in those with dominant (usually left) anterior temporal lobe dementia, due to a different, a more literal way of perceiving the world. These cases suggest the possibility that non-invasive brain stimulation, by inhibiting the left anterior temporal lobe or by diminishing left hemisphere dominance, can reduce the said ‘top-down’ mechanism, thereby enhance human ability for certain insight related tasks.

Some known methods of enhancing cognition with DC stimulation apparently aims to excite brain areas involved with certain functions in order to selectively recruit or facilitate human neural networks that are involved for a given task. For example, applying anodal DC stimulation (increase excitability) to the prefrontal cortex to enhance declarative memory (Marshall, Molle, Hallschmid, & Born, 2004), working memory (Fregni, et al., 2005; Ohn, et al., 2008), planning ability (Dockery, Hueckel-Weng, Birbaumer, & Plewnia, 2009); applying anodal DC stimulation to the right inferior frontal area to accelerate learning in detecting obscured objects (Clark, et al., 2010); applying anodal DC stimulation to the right parietal cortex to improve visual search, attention(Bolognini, Fregni, Casati, Olgiati, & Vallar, 2010), numerical competence (Cohen Kadosh, Soskic, Iuculano, Kanai, & Walsh, 2010), and spatial orientation (Bolognini, Olgiati, Rossetti, & Maravita, 2010); finally, applying anodal DC stimulation to the left posterior perisylvian region and Broca's area to improve language learning (Floel, Rosser, Michka, Knecht, & Breitenstein, 2008; Sparing, Dafotakis, Meister, Thirugnanasambandam, & Fink, 2008) and learning of artificial grammar (de Vries, et al., 2010), respectively.

The approaches described in this BACKGROUND section are approaches that could be pursued, but not necessarily approaches that have been previously conceived or pursued. Therefore, unless otherwise indicated, it should not be assumed that any of the approaches described in this section qualify as prior art merely by virtue of their inclusion in this section. Similarly, issues identified with respect to one or more approaches should not assume to have been recognized in any prior art on the basis of this section, unless otherwise indicated.

Any discussion of the background art throughout the specification should in no way be considered as an admission that such art is widely known or forms part of common general knowledge in the field.

### SUMMARY OF THE INVENTION

It is an object of the present invention to provide a method and device for increasing human ability for idea generation, creativity and insight.

In accordance with a first aspect of the present invention, there is provided a method of increasing the productive capability for user idea generation or insight related task of a user, the method comprising the steps of: a) applying an electric current stimulation to the left anterior temporal lobe of the user's brain during the task.

The electric current stimulation preferably can include a period of substantially constant electric current stimulation. In some embodiments, the current stimulation occurs by placing a cathodal electrode on the left hemisphere side of the head of the user and an anodal electrode on the right hemisphere side of the head of the user. The cathodal electrode can be placed adjacent the left anterior temporal lobe of the user's brain.

Indeed, this possibility was demonstrated for the first time by the present inventor, with a unique protocol in DC stimulation. In an embodiment, 60 healthy right-handed participants were asked to solve an unfamiliar insight problem involving matchstick arithmetic (see FIG. 4) while receiving direct current stimulation to the anterior temporal lobes (ATL). Only 20% of participants in the sham (placebo) stimulation group solved an unfamiliar insight problem, whereas 3 times as many participants did so (p=0.011) with cathodal stimulation (decreased excitability) of the left ATL together with anodal stimulation (increased excitability) of the right ATL (see FIG. 5). Hemispheric differences were found in that a stimulation montage involving the opposite polarities did not facilitate performance. Such findings are consistent with the cases described above that inhibition to the left ATL can sometimes lead to a cognitive style that is less influenced by previous experiences, leading to unusual skills.

While there has been growing studies showing that DC stimulation can incrementally enhance other aspects of cognition (Hamilton et al, 2011), the present invention of increasing human ability for insight related tasks is radically different in its conceptual paradigm, experimental protocol, and efficacy.

In contrast, one aspect of our method includes inhibiting the mind's top-down mechanism that imposes prior knowledge on the task at hand. This mitigates the ‘mental set’ effect during an insight related task. By inhibiting the said top-down mechanism, one aspect of the method of the present invention increases the chance that alternative, novel representations of the tasks at hand, often hidden from conscious awareness (for the sake of efficiency in dealing with the familiar) are considered. One aspect of our method includes temporarily enabling a person to have a qualitatively different perception of an insight related task. In some versions, an insight related includes a style of perception that is less influenced by past expectations and more receptive to novel representations. One embodiment of the present invention includes a method that includes applying the DC stimulation briefly for a specific situation. That is, one embodiment of the present invention is a method that temporarily enables an ability that, without application of the DC stimulation described herein, is otherwise extremely difficult for the normal human mind. It is theorized that this is due to a human's cognitive makeup.

In one embodiment, the DC stimulation inhibits the left anterior temporal lobe, an area that is theorized to be associated with mental templates or preconceptions (Baron, 2011; Chaumon, 2009). In one embodiment, the DC stimulation inhibits diminishes left hemisphere dominance. The inventor has carried out three studies that show that one embodiments of the method that include applying cathodal stimulation of the left anterior temporal lobe while simultaneously applying anodal stimulation of the right anterior temporal lobe, increase in a measurable way the ability for idea generation or insight related tasks in the human subject on whom the simultaneously stimulations are applied. Embodiment of the method of DC stimulation for increasing human ability for insight related tasks are consistent with theory and experimental evidence that the left hemisphere is important for processing “well routinized representations and strategies” and the right hemisphere is “critical for processing novel cognitive situations” (Goldberg 1994).

### DESCRIPTION OF THE DRAWINGS

Preferred embodiments of the present invention will now be described with reference to the accompanying drawings in which:

FIG. 1 is a schematic example of applying a DC stimulation method for increasing human ability for idea generation and insight related tasks, a cathodal cable 121 to which is coupled a cathodal electrode, an anodal cable 122 to which is coupled anodal electrodes.

FIG. 2 is a flowchart depicting an embodiment of a method for applying a DC stimulation for increasing human ability for an idea generation and/or insight related task.

FIG. 3 is an example where the stimulation method of the present invention is used to enabled solution of a so-called ‘unsolvable’ problem. A task of the problem is for a human subject to draw four straight lines that connect all nine dots n without lifting a pen from paper and without retracing any line. A majority of published studies show that statistically no participants can solve this deceptively simple problem(Kershaw and Ohlsson, 2004)—a fact the inventor confirmed by experiment. But with 10 minutes of the stimulation protocol of method embodiments of the present invention, more than 40% of healthy participants successfully solved the problem.

FIG. 4 shows an example illustrating a phenomenon that once humans have learned to solve a problem involving a kind of insight by one method, humans often have difficulties in generating solutions to problems involving a different kind of insight. Ollinger et al 2008 demonstrated by experiment that repeatedly solving problems requiring one kind of insight (e.g. changing an X to a V as shown in Type 1 of of FIG. 4) impairs subsequent performance on problems requiring a different kind of insight (e.g. changing a + sign to an = sign as shown in Type 2 in FIG. 4. Ollinger et al found that only 10% of participants could solve the Type 2 problem shown in FIG. 4 after solving a series of 27 Type 1 problems.

FIG. 5 illustrates a feature of using embodiments of the present invention. While participants in all stimulation groups had difficulties in the first minute, after 150 seconds, only those who received cathodal DC stimulation of the left anterior temporal lobe together with anodal DC stimulation of the right anterior temporal lobe (L−R+ stimulation), as described herein, improved over time. By the end of 360 seconds, 60% of those in the L−R+ stimulation group could solve the problem whereas only 20% of those in the sham (placebo) stimulation group could do so (p=0.022, two tail fisher's exact test). Stimulation with the opposite polarities (L+R−) did not lead to significant improvement in performance.

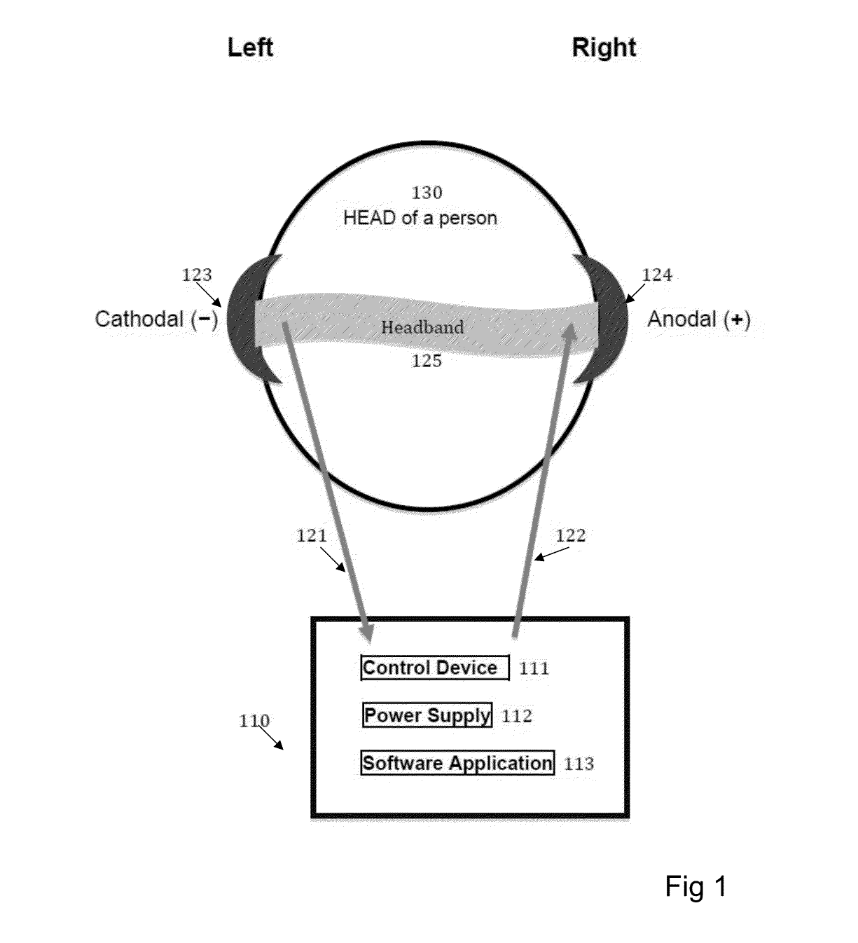

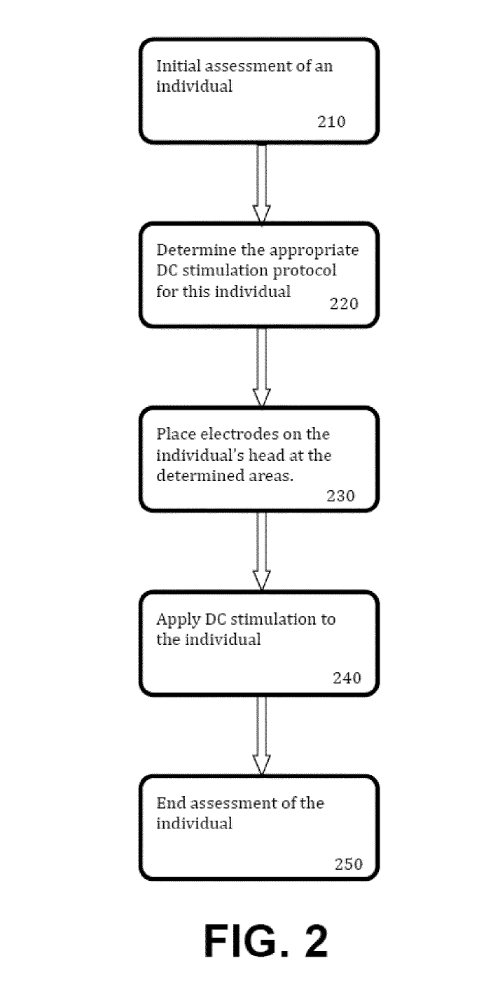

### DESCRIPTION OF EXAMPLE EMBODIMENTS

### Overview

The present invention relates to methods for increasing human ability for idea generation and insight related tasks. The method includes applying DC stimulation at selected brain regions. Tasks involving what commonly is understood to be “thinking outside the box” are difficult. Such tasks for a human subject often requires a qualitatively different perception by the human subject of the task at hand. Here are described embodiments of a method for facilitating such pursuits by applying DC stimulation at selected brain areas before or during an idea generation or insight related task. Also described are embodiments delivering direct current to a human subject in order to stimulate brain tissue in the human subject in a way that affects cortical excitability at selected brain areas. In one embodiment, the direct current modulates left hemisphere dominance in the human subject. More particularly, embodiments of the methods and devices are to temporarily reduce the human subject's mind's top-down mechanism so that the human subject can be less influenced by past expectations and more receptive to novel representations before or during idea generation or insight related tasks.

One feature of some embodiments is that the method is non-invasive for the human subject.

Embodiments of the present include increasing idea generation, creativity and insight related tasks in healthy individuals. Embodiments of the present include increasing idea generation, creativity and insight related tasks in people with brain abnormalities. Embodiments of the present include increasing idea generation and insight related tasks in people who want to be more creative. Embodiments of the present include increasing a human ability to ‘think outside the box’ during idea generation and insight related tasks. Embodiments of the present include applying the method for a situation that includes brainstorming for a new idea. Embodiments of the present include applying the method for a situation that includes an artistic pursuit. Embodiments of the present include applying the method for a situation where it is beneficial to have a fresh and/or novel representation of perceiving a task at hand.

Embodiments of the present invention include methods comprising placing one or more electrodes on the head of a particular individual related to one or more pre-defined areas of the particular individual's brain. In some embodiment, the methods also include applying a DC current to the electrodes at predefined brain areas of the individual at least one of before, during, and after an idea generation or an insight related task. In some embodiment, the methods also include controlling the application of DC stimulation by a separate control device.

In some embodiments, the methods include applying cathodal DC stimulation of the left anterior temporal lobe while simultaneously applying anodal DC stimulation of the right anterior temporal lobe, at least one of before, during and after the idea generation or the insight related task.

In yet further embodiments, the methods include methods include assessing the human subject's individual's cognitive profile, that includes one or more of the human subject's memory, reaction time, intelligence quotient (IQ), previous response to DC stimulation, and the human subject's ability for a particular insight related task in order to determine a stimulation protocol that is specific for the human subject's carrying out the idea generation or insight related task.

In yet further embodiments, the methods include delivering the DC current stimulation using a DC stimulator that is connected to a plurality electrodes, such that a first electrode delivers cathodal stimulation and a second electrode delivers anodal stimulation.

Embodiments the present invention include is a DC stimulator device that is connected to a plurality electrodes, such that a first electrode delivers cathodal stimulation to a human subject and a second electrode delivers anodal stimulation to the human subject.

Particular embodiments may provide all, some, or none of these aspects, features, or advantages. Particular embodiments may provide one or more other aspects, features, or advantages, one or more of which may be readily apparent to a person skilled in the art from the figures, descriptions, and claims herein.

### EXAMPLE 1

### Brain Stimulation Enabled the Solution of an ‘Unsolvable’ Problem

### Introduction

It is theorized and experiments have suggested that human minds are well adapted to deal with those problems that have ecological significance, even if they are computationally challenging; whereas humans consistently struggle with certain cognitive biases, even if humans are taught to avoid them (Kahneman et al., 1982).

Intriguingly, the opposite can be true for humans having an unusual mind. For example, it is theorized and experiments have suggested that certain so-called savants can perform esoteric numerical calculations while being deficient in elementary arithmetic (Snyder and Mitchell, 1999). Furthermore, it is theorized and experiments have suggested that so-called savants can have extraordinary veridical memories while struggling to get the gist of a story or recognise familiar faces (Snyder and Mitchell, 1999).

Interestingly, there is evidence that such unusual ability is associated with left hemisphere inhibition together with right hemisphere facilitation (Treffert, 2009). This inspired the inventor of the present invention to investigate whether transcranial direct current stimulation (tDCS), by diminishing left hemisphere dominance, could enable solving a problem that is extraordinarily difficult for a normal human mind.

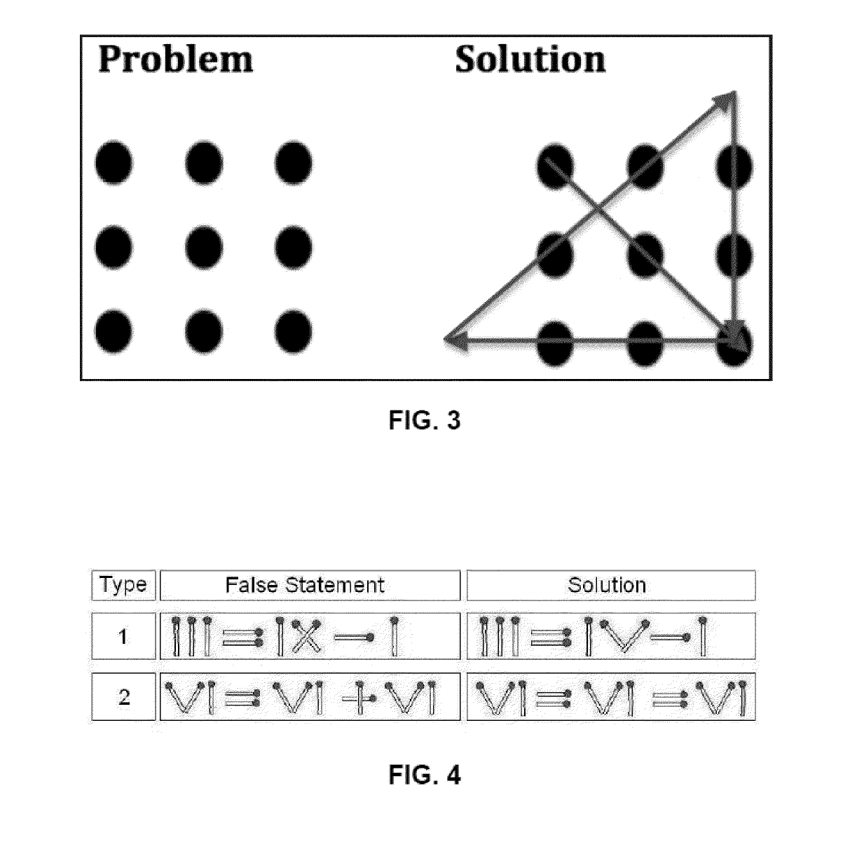

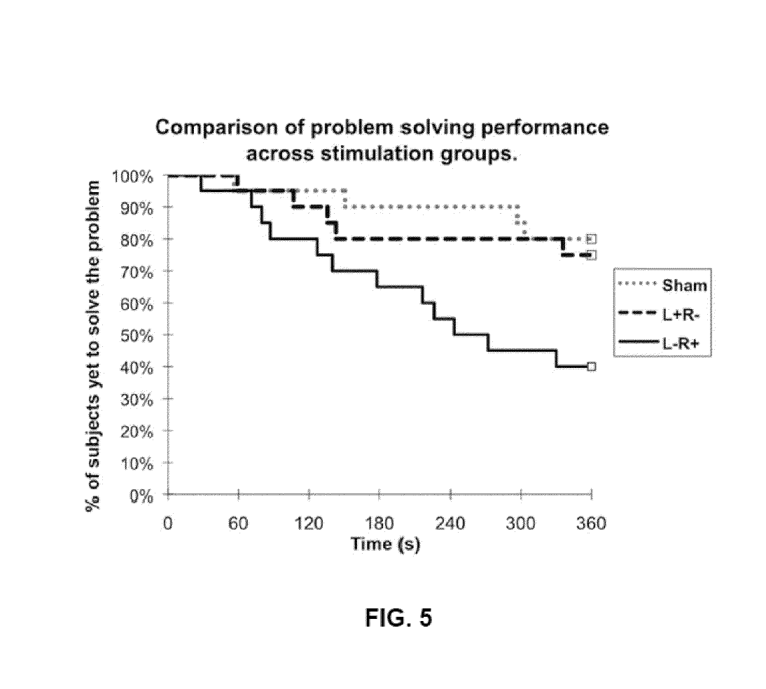

Specifically, the inventor hypothesized that cathodal stimulation (decreasing excitability) of the left anterior temporal lobe (ATL) together with anodal stimulation (increasing excitability) of the right ATL (‘L−R+’ stimulation) could enable healthy participants to solve the nine-dot problem (see FIG. 3), a problem that is commonly regarded as almost unsolvable, and it is theorized and experiments have suggested that this is due to human cognitive makeup (Kershaw and Ohlsson, 2004). The inventor's hypothesizing was based on recent evidence that applying such a tDCS protocol can significantly improve insight problem solving, possibly by mitigating the bottleneck to ‘thinking outside the box’. See (Chi & Snyder 2011), included herein as the Appendix.

### Results

The inventor carried an experiment on 22 participants.

None of the 22 participants in the main experiment solved the nine-dot problem before stimulation. But with 10 minutes of right lateralizing transcranial direct current stimulation (tDCS), more than 40% of participants could do so. Specifically, whereas none of the 11 participants in what is called a ‘sham stimulation’ control condition solved the nine-dot problem before, during or immediately after stimulation, five (four during stimulation, one immediately after stimulation) out of eleven participants solved the problem as a result of L−R+ stimulation (p=0.018, one tail Fisher's exact test).

The probability of observing this finding by chance is miniscule. Even if one makes an overly conservative assumption that the expected solution rate for the nine-dot problem was 5% (instead of 0%, which is what the inventor observed), the probability that by chance, 5 out of 11 in the L−R+ stimulation group solved the problem is less than one in ten thousand, according to analysis using the binomial distribution.

One can rule out the possibility that an unrepresentative sample explains the inventor's results. The inventor found that there is no significant difference in demographic characteristics between the two stimulation groups. The inventor also found that there is no significant difference in demographic characteristics between those people who solved the nine-dot problem and those people who could not.

The finding that an embodiment of DC stimulation method of the present invention enabled more than 40% of participants to solve the ‘unsolvable’ nine-dot problem is consistent with the inventor's pilot study which shows that whereas no one solved the nine-dot problem in the sham stimulation condition, 3 out of 7 participants in the L−R+ stimulation condition did so after stimulation. It is also strongly supported by subsequent studies where the inventor included the nine-dot problem at the end of an unrelated experiment. In fact, of all the data collected over eight months, 0 out of 29 participants in the ‘sham stimulation’ condition solved the nine-dots problem, whereas 14 out of 33 participants (naïve to the nine-dot problem) in the L−R+ stimulation condition did so.

### Discussion

The nine-dot problem is a subset of problems that, although computationally simple, are nonetheless commonly regarded as extremely difficult to solve (Kershaw and Ohlsson, 2004).

One theory of what is so is that human brains, especially the left hemisphere (Gazzaniga, 2002), are wired to interpret the world through the filters of past experience(Snyder and Mitchell, 1999). It is theorized and experiments have suggested that humans, for example, are inclined to see stars, not as discrete elements, but as constellations with meanings and narratives (Snyder et al., 2006). Similarly, It is theorized and experiments have suggested that humans are inclined to see the nine-dot as a Gestalt—a square, with imposed rigid boundaries (Kershaw and Ohlsson, 2004). Similarly, It is theorized and experiments have suggested that this mechanism is mostly unconscious (Snyder and Mitchell, 1999), and cannot be easily overridden (Kershaw and Ohlsson, 2004). Similarly, It is theorized and experiments have suggested that this allows humans to rapidly deal with familiar situations(Snyder and Mitchell, 1999) but constrains humans from ‘thinking outside the box’ (Chi and Snyder, 2011), even after humans are explicitly instructed that the solution requires humans to do just that (Weisberg and Alba, 1981; Kershaw and Ohlsson, 2004).

The inventor discovered that an atypical protocol for tDCS enabled people to solve such an inherently difficult problem. It is theorized that this finding is due to inhibiting networks associated with top down imposition of prior knowledge, knowledge that inclines humans to join the dots up within the square. In particular, it is theorized and experiments have suggested that this is achieved by inhibiting the left anterior temporal lobe, an area that is believed to filter or combine lower-level information into meaningful patterns (Baron and Osherson, 2011; Chi and Snyder, 2011). It is theorized and experiments have suggested, more generally, that this is achieved by diminishing the left hemisphere, the hemisphere that is thought to specialise for recalling or performing previously learned patterns (Gazzaniga, 2002; Goldberg, 2009; Chi and Snyder, 2011; Schambra et al., 2011).

One feature of the method of the present invention and the experiments performed to validate the method in that the method doe not aim to enhance an existing ability by exciting a specific brain region associated with that ability. Instead, embodiments of the method include a stimulation protocol that mirrors left hemisphere inhibition together with right hemisphere facilitation.

While the invention does not depend on any theory or explanation, and there could be alternative explanations, the results experimentally obtained that over 40% of participants solved the problem stand on their own. The nine-dot problem is commonly understood to be so inherently difficult that most people fail to solve the problem even if they are given hints, a long time to solve the problem, or 100 attempts to solve the problem. See, e.g., (Weisberg and Alba, 1981; Kershaw and Ohlsson, 2004). A century of research has established that “the expected solution rate for this problem under laboratory conditions is 0%” (Chronicle et al., 2001; Kershaw and Ohlsson, 2004). Even amongst those who have been shown the solution, more than one third could not reproduce the solution one week later (Weisberg and Alba, 1981). People's struggle with solving this problem appears analogous to evidence that cognitive biases are inherently difficult to avoid, even for those people who have been taught to do so (Kahneman et al., 1982). The inventor' findings suggest that our stimulation protocol may be a method of mitigating some of the cognitive biases discovered by Kahneman and colleagues (1982).

Embodiments of tDCS, as described herein, enable the solution of 9 dot problem in humans. Embodiments of tDCS, as described herein are applicable to dealing with a broader class of tasks that, although deceptively simple, are nonetheless extremely difficult due to human cognitive makeup.

### Materials and Methods

### Participants

From a pilot study (see below), it was determined that the most efficient experimental design for testing the hypothesis that tDCS as applied herein, for increasing human ability for idea generation and insight related tasks, with a 95% statistical power, was to have 2 stimulation groups in the main experiment (‘Sham stimulation’ for placebo control versus ‘L−R+’ stimulation), each with 11 subjects. Twenty eight healthy right-handed aged between 19 and 63 years from the University of Sydney, Australia community participants were recruited, with six participants excluded due to previous experience with the nine dots problem. Participants were also screened and excluded if they had any neuropsychiatric disorder, current or past history of drug use, were taking any medication acting on the central nervous system or were pregnant. None of the participants experienced adverse effects as a result of tDCS.

Transcranial Direct Current Stimulation (tDCS)

tDCS involves applying a weak direct current to the scalp via two saline-soaked sponge electrodes, thereby polarizing the underlying brain tissue with electrical fields (Nitsche et al., 2008). Although it is controversial how focal the effects of tDCS is (Datta et al., 2009; Sadleir et al., 2010), recent neuroimaging studies (Paquette et al., 2011; Zheng et al., 2011) demonstrate that tDCS does modulate cortical excitability and changes in cerebral blood flow at the stimulated region under the electrodes.

One embodiment of the method used in the experiment used a custom made, battery-driven, constant current stimulator with a maximum output of 2 mA and 2 sponge electrodes each with an area of 35 cm2.

For the active (L−R+ stimulation) condition, a constant current of 1.6 mA intensity was applied, and was manually and slowly ramped up (over 30 seconds). Specifically, for L−R+ stimulation, cathodal tDCS at the left anterior temporal lobe (ATL) together with anodal tDCS at the right ATL was applied for approximately 10 minutes, which is believed to affect cortical excitability for about an hour (Nitsche et al., 2008). The stimulation protocol used in one embodiment of the present invention. is critically discussed in Chi and Snyder (2011), included herein as the Appendix. It is consistent with recent modelling and behavioural evidence that bilateral tDCS at the temporal lobes is effective for modulating hemispheric dominance (Turkeltaub et al., 2011), leading to left lateralisation and an enhanced reading ability.

For the sham stimulation (placebo control) condition, the sponge electrodes were placed in the same positions as in active stimulation, but after 30 seconds, the electrical current was covertly ramped down to off. This procedure is known to produce sensations virtually indistinguishable from that in the active condition(Gandiga et al., 2006; Nitsche et al., 2008).

### Procedure and Tasks

Participants were given a total of 9 minutes to solve the nine-dot problem (Chronicle et al., 2001; Kershaw and Ohlsson, 2004): 3 minutes ‘before’ stimulation, 3 minutes (‘online’) during stimulation and 3 additional minutes (‘offline’) immediately after the stimulator was turned off.

All task stimuli were shown on a 21.5 inch Apple IMac computer screen at a viewing distance of approximately 30 ft. However, participants were asked to attempt the nine-dot problem by pen and paper and were allowed unlimited attempts for 3 minutes to solve the problem.

Participants were also given a simple arithmetic control task each time prior to attempting the nine-dot problem. This task involved answering as many 2-digit addition arithmetic problems (e.g. 51+23) as possible in 2 minutes. Its score (the number of correct answers) provides an indication of reaction time and quantitative ability. Participants were required to type in the right answer on the keyboard before they could move on to the next problem.

During the ‘online’ phase, participants were given the control task and the nine-dot problem only after they have received five minutes of tDCS. This was to ensure that there was sufficient change in cortical excitability (Nitsche et al., 2008). During this five-minute period, participants were asked to do a distraction task where they had to spot the one difference between two very similar pictures, by clicking at the difference on the computer screen.

### Statistical Analysis

Analysis is based on Chi and Snyder 2011, and also the results of a pilot study. A one tailed Fisher's exact test is used to compare the solution rate between the sham stimulation group and the L−R+ stimulation group. Additionally, the binomial distribution is used to compare the observed solution rate in the L−R+ stimulation group and the expected solution rate for the nine-dot problem (the analysis used an overly conservative estimate of 5% —the majority of studies reported a solution rate of 0%, although there have been a few studies reporting a solution rate of 5%).

### Pilot Study

Prior to the experiment reported herein, a pilot study was performed in order to determine the necessary sample size and the experimental design. The stimulation protocol described in Chi and Snyder (2011) was used, with 7 participants in each of the three different stimulation conditions (‘L−R+ stimulation’, stimulation of the opposite polarities, that is ‘L+R− stimulation’, and ‘Sham stimulation’). As in the current study, participants were given a total of 9 minutes before, during and after stimulation to solve the problem. It was found that three out of seven subjects who failed to solve the nine-dot problem before stimulation became successful as a result of ‘L−R′’ stimulation. In contrast, no one in the L+R− stimulation group (stimulation of the opposite polarities) nor the sham stimulation group could solve the problem before, during or after stimulation. This confirms the findings described in Chi and Snyder (2011) (see Appendix) that only L−R+ stimulation (not L+R− stimulation nor Sham stimulation) could improve performance for an insight problem. It also supports the evidence that alertness is unlikely to be a factor, since stimulation of the opposite polarities (L+R− stimulation) did not affect performance.

Creativity is often defined as the ability to generate novel and appropriate ideas. That is, an essential element of creative potential is the ability to transcend the familiar and produce novel ideas.

### EXAMPLE 2

### Brain Stimulation Further Enables Solution to Creative Problem

In a further experiment, we chose to ask participants to provide uses for a piece of paper or string, both common objects that are highly familiar and accessible to all. This divergent thinking task is a common test of creative potential (Snyder et al, 2004 “The Creativity Quotient: An objective scoring of ideational fluency)

By testing 24 subjects, it was found that those people in the sample of active tDCS condition produced approximately twice as many new ideas as the people in a sham stimulation condition, both during stimulation (13.8 vs 7.3 uses, p=0.002) and immediately after stimulation (9.5 vs 4.3 uses, p=0.017). In other words, those people who received the stimulation protocol had an average total of 23.3 ideas during and immediately after stimulation whereas people who received sham stimulation only had 11.6 ideas.

The results here again confirm that the stimulation protocol (by placing the cathodal electrode on the left anterior temporal lobe and placing the anodal electrode on the right anterior temporal lobe) can enhance people's ability for idea generation, insight and creativity related tasks.

### EXAMPLE 3

### Brain Stimulation Further Enables Solution to Creative Thinking Problem

Torrance tests of creative thinking (TTCT) is one of the most established test for creativity. The term ‘creative thinking abilities’ as used in the Torrance tests, ‘refers to that constellation of generalized mental abilities that is commonly presumed to be bought into play in creative achievement.’ (Torrance, Ball & Safter, 1992). Although high scores on the TTCT does not guarantee creative behaviors, the authors of the test found that “scores on the TTCT taken during the high school years correlate about 0.51 with adult creative achievement twelve years later”.

Various dimensions of creativity can be scored using the TTCT, especially fluency (the number of relevant ideas, in this case, figural images), originality (the number of statistically infrequent or unusual figural images, relative to the norm). In addition to fluency and originality, the results can also be analysed in light of ‘resistance to premature closure’ (how open-ended the figures are), an indication of open-mindness and incubation ability (TORRANCE, 1979).

In the third example, the standard scoring guide ‘Torrance Tests of Creative Thinking: Streamlined Scoring Guide Figural A and B’ (1992) by Torrance, Ball & Safter. Participants were asked to complete activity 2 and activity 3 of the ‘Figural Booklet A and B’ in the TTCT. 12 participants were asked to complete activity 2 and activity 3 twice. Of the 5 people who received active transcranial direct current stimulation (the same protocol as described above), they were asked to complete activity 2 and 3 of the TTCT once before stimulation and once during stimulation, with approximately 10 minutes break between each testing session. Of the 7 people who did not receive stimulation, they were asked to do activity 2 and 3 of the TTCT twice with approximately 10 minutes break between each testing session.

It was found that using the same stimulation protocol (cathodal electrode on the left anterior temporal lobe and anodal electrode on the right anterior temporal lobe) it was possible to increase people's ideational fluency (the number of figures produced) by 40% (ANOVA, p=0.02). In particularly, those people in the no stimulation condition completed 24 figures before stimulation and 24 figures the second time. In contrast, those people who received the active stimulation protocol increased the number of figures they produced from 16 to 21.

In addition, it was also found that a 50% improvement (p=0.02, ANOVA) occurred in the scores of originality, as a result of our active stimulation protocol. In particular, those who received the active stimulation protocol improved their points for originality from 10.4 to 15.6 (p=0.009, t-test). In contrast, those who received no stimulation showed no improvement the second time they performed activity 2 and activity 3 of the TTCT (a change from 18.5 points to 18.4 points, p=0.96, t-test).

Finally, it was found that the stimulation protocol can also improve (p=0.022, ANOVA) a person's score for ‘resistance to premature closure’ (how open ended the figures are). Those who received the active stimulation protocol showed an improvement from a score of 6 to 11.2, an improvement of 87% (p=0.003, t-test). In contrast, there is no change (from a score of 12.1 to 12.43, p=0.88, t-test) in the score of ‘resistance to premature closure’ in those people who received no stimulation. This suggest that the stimulation protocol can facilitate open mindness and the incubation process. [TORRANCE, E. P. (1979), Resistance to Premature Gestalt Closure as a Possible Indicator of Incubation Ability]

To sum up, in this example experiment, it was found that our stimulation protocol enhanced a person's ability for fluency (the number of ideas), originality (how unusual the ideas are), and ‘resistance to premature closure’ (how open ended the ideas are), using the Torrance tests of creative thinking, a well-established test of creative potential.

### EXAMPLE 4

### Enabling the Solution of a Difficult Brainteaser—the ‘Cake’ Problem, by the Stimulation Protocol

In a fourth example, the problem was posed of: “How can a cake be cut into eight pieces of equal size with only three cuts?”. Most people find it easy to dissect a cylinder into eight equal parts. However, if the cylinder is disguised as a cake, then the majority of cannot solve this problem because they assume that a cake can only be cut from the top, instead of from the middle. The cake problem is an example where our past experiences can inhibit our ability to deal with a problem. (see Segal, E. (2004)).

In the present example, the participants were assigned to either the active stimulation group (cathodal electrode on the left anterior temporal lobe and anodal electrode on the right anterior temporal lobe) or the sham (placebo) stimulation group. They are asked to solve the cake problem 3 times, once before stimulation, once during stimulation and once immediately after stimulation.

Of those 7 participants in the sham (placebo) stimulation group who could not solve the cake problem before stimulation, no one solve the problem during or immediately after stimulation. In contrast, of the 10 people in the active stimulation group who could not solve the cake problem before stimulation, 2 of them did so during stimulation. The results suggest that our stimulation protocol, perhaps by inhibiting false assumptions, can facilitate incubation or insight in brainteasers.

### General Interpretation

Unless specifically stated otherwise, as apparent from the following description, it is appreciated that throughout the specification discussions utilizing terms such as “processing,” “computing,” “calculating,” “determining” or the like, may refer to, without limitation, the action and/or processes of hardware, e.g., an electronic circuit, a computer or computing system, or similar electronic computing device, that manipulate and/or transform data represented as physical, such as electronic, quantities into other data similarly represented as physical quantities.

Note that when a method is described that includes several elements, e.g., several steps, no ordering of such elements, e.g., of such steps is implied, unless specifically stated.

Reference throughout this specification to “one embodiment,” “an embodiment,” “some embodiments,” or “embodiments” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the above description of example embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the DESCRIPTION OF EXAMPLE EMBODIMENTS are hereby expressly incorporated into this DESCRIPTION OF EXAMPLE EMBODIMENTS, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description provided herein, numerous specific details are set forth. However, it is understood that embodiments of the invention may be practiced without these specific details. In other instances, well-known methods, structures and techniques have not been shown in detail in order not to obscure an understanding of this description.

As used herein, unless otherwise specified, the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

Any discussion of other art in this specification should in no way be considered an admission that such art is widely known, is publicly known, or forms part of the general knowledge in the field at the time of invention.

In the claims below and the description herein, any one of the terms comprising, comprised of or which comprises is an open term that means including at least the elements/features that follow, but not excluding others. Thus, the term comprising, when used in the claims, should not be interpreted as being limitative to the means or elements or steps listed thereafter. For example, the scope of the expression a device comprising A and B should not be limited to devices consisting of only elements A and B. Any one of the terms including or which includes or that includes as used herein is also an open term that also means including at least the elements/features that follow the term, but not excluding others. Thus, including is synonymous with and means comprising.

Similarly, it is to be noticed that the term coupled, when used in the claims, should not be interpreted as being limitative to direct connections only. The terms “coupled” and “connected,” along with their derivatives, may be used. It should be understood that these terms are not intended as synonyms for each other, but may be. Thus, the scope of the expression “a device A coupled to a device B” should not be limited to devices or systems wherein an input or output of device A is directly connected to an output or input of device B. It means that there exists a path between device A and device B which may be a path including other devices or means in between. Furthermore, coupled to does not imply direction. Hence, the expression “a device A is coupled to a device B” may be synonymous with the expression “a device B is coupled to a device A.” “Coupled” may mean that two or more elements are either in direct physical or electrical contact, or that two or more elements are not in direct contact with each other but yet still co-operate or interact with each other.

In addition, use of the “a” or “an” are used to describe elements and components of the embodiments herein. This is done merely for convenience and to give a general sense of the invention. This description should be read to include one or at least one and the singular also includes the plural unless it is obvious that it is meant otherwise.

Thus, while there has been described what are believed to be the preferred embodiments of the invention, those skilled in the art will recognize that other and further modifications may be made thereto without departing from the spirit of the invention, and it is intended to claim all such changes and modifications as fall within the scope of the invention, to the extent permitted by law. For example, to the extent permitted by law: any formulas given above are merely representative of procedures that may be used; functionality may be added or deleted from the block diagrams and operations may be interchanged among functional blocks; and steps may be added to or deleted from methods described within the scope of the present invention.

We claim:

1. A method of increasing the productive capability for user idea generation, creativity or insight related task of a user, the method comprising the steps of:
a) applying an electric current stimulation to the left anterior temporal lobe of the user's brain during the task.

2. A method as claimed in claim 1 wherein said electric current stimulation includes a period of substantially constant electric current stimulation.

3. A method as claimed in claim 1 wherein said current stimulation occurs by placing a cathodal electrode on the left hemisphere side of the head of the user and an anodal electrode on the right hemisphere side of the head of the user.

4. A method as claimed in claim 3 wherein the cathodal electrode is placed adjacent the left anterior temporal lobe of the user's brain.

5. A method as claimed in claim 3 wherein the anodal electrode is placed adjacent the right anterior temporal lobe of the user's brain.

6. A method as claimed in claim 3 wherein the level of current simulation is substantially from 0.5 mA to 2.5 mA.

7. A method as claimed in claim 3 wherein electrical current stimulation is applied before, during or after a user performs idea generation, creativity or insight related tasks.

8. A method as claimed in claim 3 wherein electrical current stimulation involves the use of transcranial direct current stimulation or transcranial random noise stimulation to affect cortical excitability or neural activity.

9. A method of increasing human ability for idea generation or insight related tasks, the method comprising the steps of:
a) assessing one or more parameters related to an individual's cognitive profile, or the individual's ability for insight related tasks so as to determine a stimulation protocol specific for a particular individual carrying out an idea generation or insight related task;
b) placing one or more electrodes on the head of the particular individual related to one or more pre-defined areas of the particular individual's brain;
c) applying an DC current to the electrodes to stimulate brain tissue in the particular individual's brain, at least one of the pre-defined brain areas;
d) controlling the application of the DC current applied to the specific pre-defined brain areas during at least one of before, during or after such an idea generation or insight related task.

10. The method of claim 9, wherein said stimulating brain tissue includes at least one of modulating cortical excitability, modulating left hemisphere dominance, modulating right hemisphere dominance or upregulation or downregulation of neural activities.

11. The method of claim 9, wherein said assessing includes determining the appropriate stimulation protocol according to an individual's cognitive profile or previous responses to electrical current stimulation.

12. The method of claim 9, wherein said assessing further includes assessing the human subject to determine if said human ability for idea generation or insight related tasks has been improved over time in the human subject and what further electrical current stimulation is desired.

13. The method of claim 9, wherein said cognitive profile includes an individual's performance in at least one of reaction time, memory, aspects of the intelligence quotient (IQ), mental health, and previous responses to electrical current stimulation.

14. The method of claim 9, wherein said applying includes applying at least one of cathodal stimulation of the left temporal lobe, and anodal stimulation of the right temporal lobe at a time that is at least one of before, during, and after the idea generation or insight related tasks.

15. The method of claim 9, wherein said applying includes applying cathodal stimulation of the left anterior temporal lobe while simultaneously applying anodal stimulation of the right anterior temporal lobe, at least before, during, and after the idea generation or insight related tasks.

16. The method of claim 9, wherein said applying includes applying a DC current to at least one of the pre-defined brain areas in the range of from 0 to <2 mA at least one of before, during, and after the idea generation or insight related tasks.

17. The method of claim 9, wherein said controlling includes selecting whether the stimulation is anodal or cathodal stimulation, selecting the duration, location, and intensity of the stimulation current at least one of before, during, and after the idea generation or insight related tasks.

18. The method of claim 9, wherein the electrodes are located at specific brain areas so that the application of the DC current increases the particular individual's ability to perceive in a way that is less-top down, less influenced by past expectations, at least one of before, during or after idea generation or insight related tasks.

19. The method of claim 1, wherein the idea generation, creativity or insight related tasks includes one of brainstorming for new ideas, problem solving or any other artistic pursuits.

20. The method of claim 1, wherein increasing the productive capability for user idea generation, creativity or insight related task of a user includes increasing human ability to avoid cognitive biases related to mental fixation, the mental set effect or confirmation bias.
